(19) United States
(12) Patent Application Publication
(10) Pub. No.: US 2021/0290306 A1
(43) Pub. Date: Sep. 23, 2021

(54) MEDICAL INFORMATION MANAGEMENT SERVER, SURGERY TRAINING DEVICE, SURGERY TRAINING SYSTEM, IMAGE TRANSMISSION METHOD, DISPLAY METHOD, AND COMPUTER READABLE RECORDING MEDIUM

(71) Applicant: Sony Olympus Medical Solutions Inc., Tokyo (JP)

(72) Inventor: Kiyoshi ICHIKAWA, Tokyo (JP)

(73) Assignee: Sony Olympus Medical Solutions Inc., Tokyo (JP)

(21) Appl. No.: 17/184,610

(22) Filed: Feb. 25, 2021

(30) Foreign Application Priority Data
Mar. 19, 2020 (JP) 2020-048703
Dec. 24, 2020 (JP) 2020-215826

(51) Int. Cl. A61B 34/10; G06T 7/70; G06F 3/14; G06T 7/00; G16H 20/40; G16H 70/20; G16H 30/40; G09B 9/00

(52) U.S. Cl. CPC A61B 34/10; G06T 7/70; G06F 3/14; G06T 7/0012; G16H 20/40; A61B 2034/105; G16H 30/40; G09B 9/00; G06T 2207/10016; G06T 2207/30004; A61B 2034/104; G16H 70/20

(57) ABSTRACT

A medical information management server includes: a recording unit configured to record at least a plurality of sets of operative procedures in a corresponding manner to a plurality of sets of simulation data containing data related at least to body tissues; and a control unit configured to, in a case where one of the plurality of sets of operative procedures is selected at a client side, send simulation data corresponding to the selected operative procedure to the client side.

### CROSS-REFERENCE TO RELATED APPLICATIONS

This application claims priority from Japanese Application No. 2020-048703, filed on Mar. 19, 2020 and Japanese Application No. 2020-215826, filed on Dec. 24, 2020, the contents of each are incorporated by reference herein in its entirety.

### BACKGROUND

The present disclosure relates to a medical information management server, a surgery training device, a surgery training system, an image transmission method, a display method, and a computer readable recording medium.

In the related art, as far as a surgery training device is concerned, a technique is known that is reproduced during the display at the time of practicing the visual performance of an educational video accompanying the movement of an endoscope (for example, refer to Japanese Patent Application Laid-open No. 2016-148765). In that technique, the movement history of an educational endoscope during the process of taking an educational video using that educational endoscope is reproduced in a practice endoscope that is inserted in a practice device which provides a surgery practice space therein; and then the educational video and the video taken by the practice endoscope are synthesized and displayed.

### SUMMARY

Meanwhile, in order to become a good practitioner, a medical student or a medical intern needs to be present at a large number of actual surgeries and cases so as to gain experience of various surgeries. However, in Japanese Patent Application Laid-open No. 2016-14876 mentioned above, merely the movement history of a single educational endoscope is reproduced using a practice endoscope. Hence, not only it is not possible to deal with various surgeries and cases, but it is also not possible for a medical student or a medical intern to trace the procedure performed by a practitioner or a professor or to attempt a procedure according to some different approach.

According to one aspect of the present disclosure, there is provided a medical information management server including: a recording unit configured to record at least a plurality of sets of operative procedures in a corresponding manner to a plurality of sets of simulation data containing data related at least to body tissues; and a control unit configured to, in a case where one of the plurality of sets of operative procedures is selected at a client side, send simulation data corresponding to the selected operative procedure to the client side.

According to another aspect of the present disclosure, there is provided a surgery training device including: a display unit configured to display a simulation video based on simulation data sent from a medical information management server which records a plurality of operative procedures in a corresponding manner to a plurality of sets of simulation data containing data related at least to body tissues; a medical apparatus; a detecting unit configured to detect operation details of operation performed by an operator with respect to the medical apparatus; and a control unit configured to display, in the display unit, medical apparatus images, which are related to the medical apparatuses, on the simulation video, wherein based on the operation details detected by the detecting unit, the control unit is configured to control display form of the medical apparatus images and the simulation video.

### BRIEF DESCRIPTION OF THE DRAWINGS

FIG. 1 is a diagram illustrating a schematic configuration of a surgery training system according to an embodiment;

FIG. 2 is a block diagram illustrating the functional configuration of the main parts of a surgery training device according to the embodiment;

FIG. 3 is a block diagram illustrating a functional configuration of a medical information management server according to the embodiment;

FIG. 4 illustrates an example of a table storing correspondence between a plurality of operative procedures and a plurality of sets of simulation data;

FIG. 5 illustrates an example of a table storing correspondence between user IDs and levels of the surgery training of the operators U1 and training details;

FIGS. 6 to 11 are diagrams illustrating examples of setting screens displayed in a display device according to the embodiment during surgery training;

FIG. 12 is a flowchart for explaining the overview of the operations performed in the medical information management server according to the embodiment;

FIG. 13 is a flowchart for explaining the overview of a simulation data transmission operation illustrated in FIG. 12;

FIG. 14 is a flowchart for explaining the overview of a simulation data generation operation illustrated in FIG. 12; and

FIG. 15 is a flowchart for explaining the overview of the operations performed in the surgery training device according to the embodiment.

### DETAILED DESCRIPTION

An exemplary embodiment of the disclosure is described below in detail with reference to the drawings. However, the disclosure is not limited by the embodiment described below. Moreover, in the following explanation given with reference to the drawings; the shapes, the sizes, and the positional relationships are schematically illustrated only to the extent of enabling understanding of the details of the disclosure. That is, the disclosure is not limited to the shapes, the sizes, and the positional relationships illustrated in the drawings. Moreover, in the drawings, the same constituent elements are referred to by the same reference numerals. Furthermore, as an example of a surgery training system according to the disclosure, the explanation is given about a surgery training system in which a laparoscope-based endoscope system is used. However, the disclosure is applicable even in a surgery training system in which a surgical microscope system is used.

Schematic Configuration of Surgical Training System

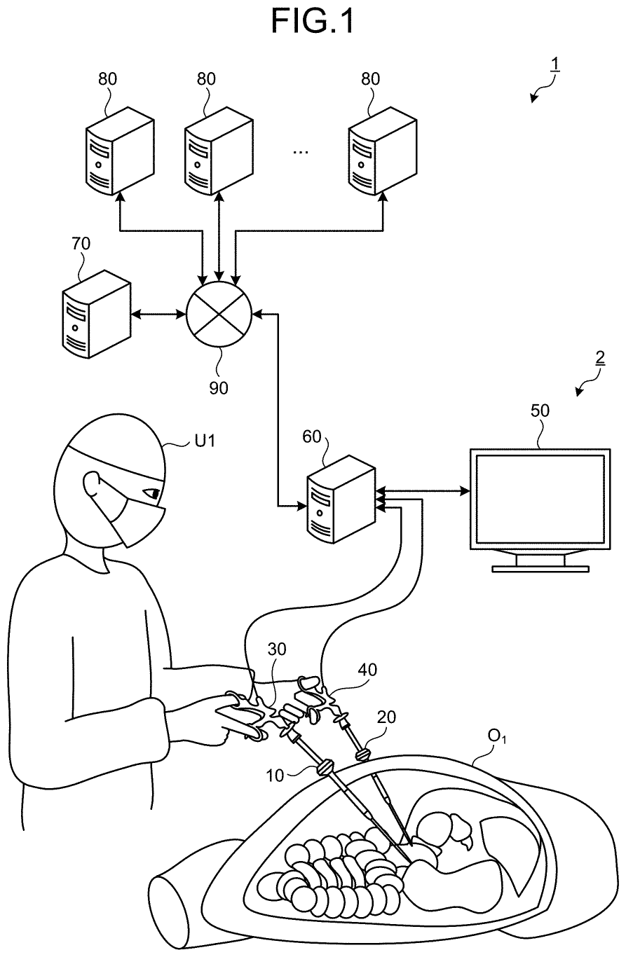

FIG. 1 is a diagram illustrating a schematic configuration of the surgery training system according to an embodiment. In a surgery training system 1 illustrated in FIG. 1, trocars are used to form a plurality of holes in the abdominal part of a model O₁ of a subject such as a person or an animal and, in a state in which the abdominal part of the model O₁ is inflated using carbon dioxide, an operator U1 (trainee) receives training of a laparoscopic surgery while inserting one or more medical apparatuses such as a medical imaging device (a videoscope such as a rigid endoscope), forceps, and an energy device such as an electrocautery scalpel; and while performing observation and procedures. With reference to FIG. 1, two holes are formed in the model O₁. However, the number of holes may be appropriately varied depending on, for example, the case, the surgical technique, and the operative procedure to be learnt in the training.

The surgery training system 1 illustrated in FIG. 1 includes trocars 10 and 20, medical apparatuses 30 and 40, a display device 50, a control device 60, a medical information management server 70, and a plurality of hospital management servers 80. Meanwhile, according to an embodiment, the trocars 10 and 20, the medical apparatuses 30 and 40, the display device 50, and the control device 60 function as a surgery training device 2.

The trocars 10 and 20 are tubular in shape and are inserted in the model O₁. In the trocars 10 and 20, the medical apparatuses 30 and 40 (described later) are respectively inserted.

The medical apparatus 30 is inserted in the model O₁ via the trocar 10. The medical apparatus 30 performs simulated procedures on the observation target in accordance with the operations performed by the practitioner such as a doctor or a medical intern. Moreover, the medical apparatus 30 detects the details of the operations performed by the practitioner and outputs the detection result to the control device 60 via a transmission cable. Examples of the medical apparatus 30 include an injection, forceps, a knife, a high-frequency snare, a treatment tool, and an energy device such as a high-frequency scalpel or an electrocautery scalpel.

The medical apparatus 40 is inserted in the model O₁ via the trocar 20. The medical apparatus 40 performs simulated procedures on the observation target in accordance with the operations performed by the practitioner such as a doctor or a medical intern. Moreover, the medical apparatus 40 outputs the details of the operations performed by the practitioner to the control device 60 via a transmission cable. Examples of the medical apparatuses 40 include an injection, forceps, a knife, a high-frequency snare, a treatment tool, and an energy device such as a high-frequency scalpel or an electrocautery scalpel.

The display device 50 displays, under the control of the control device 60, two-dimensional images and three-dimensional images based on the image data input from the control device 60. The display device 50 is implemented using, for example, a liquid crystal display or an organic EL display (organic electroluminescence display) and an audio speaker.

The control device 60 controls the constituent elements of the surgery training device 2. The control device 60 performs a variety of image processing with respect to simulation data that is input from the medical information management server 70 via a network 90; outputs the processed simulation data; and displays a simulation video based on the simulation data in the display device 50. The control device 60 is implemented using hardware such as a memory, a central processing unit (CPU), a graphics processing unit (GPU), and a field-programmable gate array (FPGA). Herein, the simulation data represents data that enables surgery simulation in a three-dimensional space including internal organs, and contains data related to an internal organ or medical apparatuses. For the internal organ and each medical apparatus, the simulation data contains, for example, the parameters given below and is defined by polygonal data in the three-dimensional space.

(1) position (the position of the internal organ (coordinate information))

(2) shape (the shape of the internal organ, the shape of each medical apparatus)

Moreover, the simulation data for the internal organ and each medical apparatus may contain, for example, parameters (3) to (7) given below.

(3) mass density

(4) stiffness

(5) friction

(6) viscosity

(7) tactile sensibility

Moreover, the simulation data may further contain information about the time series variation related to the temporal movements or the temporal deformations of the medical apparatuses or the internal organ.

The medical information management server 70 sends simulation data, which is requested by the control device 60, via the network 90. Moreover, the medical information management server 70 records two-dimensional image data and three-dimensional image data that is obtained from each of a plurality of patients using a CT (Computed Tomography) device or an MRI (Magnetic Resonance Imaging) device and that at least contains data related to the internal organ. Furthermore, the medical information management server 70 records simulation image data at least either based on intra-operative video data captured by a medical imaging device during the surgery of the subject or based on two-dimensional image data and three-dimensional image data that at least contains data related to the internal organ captured by a CT device or an MRI device. The medical information management server 70 is implemented using a processor that includes hardware such as a CPU, a GPU, and an FPGA; and using a hard disk drive (HDD) and a solid state drive (SSD).

The hospital management servers 80 send, to the medical information management server 70 via the network 90, educational data meant to be used in the simulation data generated in the medical information management server 70. The educational data represents video files in which the following information is associated to each other: two-dimensional image data or three-dimensional image data obtained from each of a plurality of subjects (patients) using a CT device or an MRI device; intra-operative video data containing at least the internal organ (for example, the stomach or the intestine) captured by a medical imaging device (for example, a video microscope such as an endoscope or a rigid endoscope) during the surgery of each of a plurality of subjects (patients); subject information (patient information) related to each of a plurality of subjects; a plurality of operating surgeons (practitioners or hospital names) that performs surgeries on a plurality of subjects; procedure information related to the operative procedures implemented on a plurality of subjects; and medical apparatus information related to each of a plurality of medical devices used in the operative procedures. Each hospital management server 80 is implemented using a processor that includes hardware such as a CPU, a GPU, and an FPGA; and using an HDD and an SSD.

Functional Configuration of Main Parts of Surgery Training Device

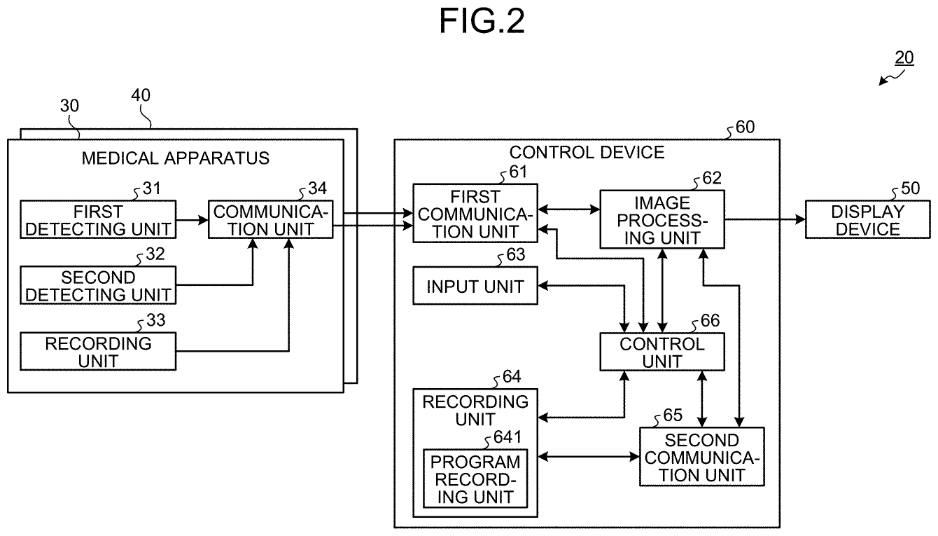

Given below is the explanation of the functional configuration of the main parts of the surgery training device 2. FIG. 2 is a block diagram illustrating the functional configuration of the main parts of the surgery training device 2.

Configuration of Medical Apparatuses

Firstly, the explanation is given about a configuration of the medical apparatus 30. The medical apparatus 30 includes a first detecting unit 31, a second detecting unit 32, a recording unit 33, and a communication unit 34. The medical apparatus 40 has an identical configuration to the configuration of the medical apparatus 30. Hence, the detailed explanation thereof is not given.

The first detecting unit 31 detects the insertion and removal of the medical apparatus 30, which is held by a practitioner, with respect to the trocar 10, such as detects the amount of pressing in of the medical apparatus 30 or the amount of drawing out of the medical apparatus 30; and outputs the detection result to the communication unit 34. The first detecting unit 31 is implemented using, for example, a gyro sensor and an acceleration sensor.

The second detecting unit 32 detects the pressure value (the pressing force value) or the speed accompanying the opening-closing operation of the medical apparatus 30 that is operated by the practitioner, and outputs the detection result to the communication unit 34. The second detecting unit 32 is implemented using a pressure sensor, a gyro sensor, and an acceleration sensor.

The recording unit 33 is implemented using a volatile memory or a nonvolatile memory, and is used to record medical apparatus information related to the medical apparatus 30. Herein, the medical apparatus information contains following information of the medical apparatus 30: model name, shape, part number, type information (for example, the type such as forceps or an electrocautery scalpel), and manufacturing date. The recording unit 33 outputs the medical apparatus information to the communication unit 34.

The communication unit 34 is capable of communicating with a first communication unit 61; and sends, to the control device 60, the detection result obtained by the first detecting unit 31, the detection result obtained by the second detecting unit 32, and the medical apparatus information recorded in the recording unit 33. The communication unit 34 is implemented using a communication module.

Given below is the explanation of a configuration of the control device 60. The control device 60 includes the first communication unit 61, an image processing unit 62, an input unit 63, a recording unit 64, a second communication unit 65, and a control unit 66.

Under the control of the control unit 66, the first communication unit 61 sends a variety of control data to the medical apparatuses 30 and 40, and outputs a variety of data received from the medical apparatuses 30 and 40 and the medical information management server 70 to the image processing unit 62 or the control unit 66. The first communication unit 61 is implemented using, for example, an S/P conversion circuit and a communication module. Moreover, the first communication unit 61 may send a variety of data to the medical apparatuses 30 and 40 using wireless communication or optical communication.

Under the control of the control unit 66, the image processing unit 62 displays a simulation video based on the simulation data (polygonal video data) input from the medical information management server 70 via the first communication unit 61. More particularly, the image processing unit 62 generates a simulation video of the observation target inside the subject (a simulation video obtained by performing graphic processing on the polygonal video data), and outputs the simulation video to the display device 50 for display purposes. The image processing unit 62 is implemented using a processor that includes hardware such as a memory, a graphics processing unit (GPU), and an FPGA.

The input unit 63 receives input of a user operation and outputs signals according to the received operation to the control unit 66. The input unit 63 is implemented using a mouse, a keyboard, a touch-sensitive panel, switches, or buttons.

The recording unit 64 is used to record a variety of data related to the surgery training device 2. The recording unit 64 includes a program recording unit 641 that is used to record various programs executed by the surgery training device 2. The recording unit 64 is implemented using a random access memory (RAM), a read only memory (ROM), a hard disk drive (HDD), or a solid state drive (SSD).

Under the control of the control unit 66, the second communication unit 65 obtains the simulation data from the medical information management server 70 via the network 90, and outputs the obtained simulation data to the control unit 66 and the image processing unit 62. The second communication unit 65 may be implemented using a communication module.

The control unit 66 controls the constituent elements of the control device 60. The control unit 66 displays, in the display device 50, medical apparatus images, which are related to the medical apparatuses, on the simulation video that is based on the simulation data obtained from the medical information management server 70 via the second communication unit 65. More particularly, based on the operation details of the medical apparatus 30 as detected by the first detecting unit 31 and the second detecting unit 32, the control unit 66 controls the display form of the medical apparatus images and the simulation video displayed in the display device 50. Moreover, when a termination signal meant for stopping the simulation video that is being displayed in the display device 50 is input from the input unit 63, the control unit 66 changes the viewpoint of the simulation video based on the operation details detected by the first detecting unit 31 and the second detecting unit 32. More particularly, in the display device 50, the control unit 66 displays a lower layer of the simulation video or displays the simulation video having the viewpoint rotated by 90° to the right side from the current visual field area. The control unit 66 is implemented using a processor that includes hardware such as a memory and a CPU. Meanwhile, according to the embodiments, the control unit 66 functions as a second control unit.

Configuration of Medical Information Management Server

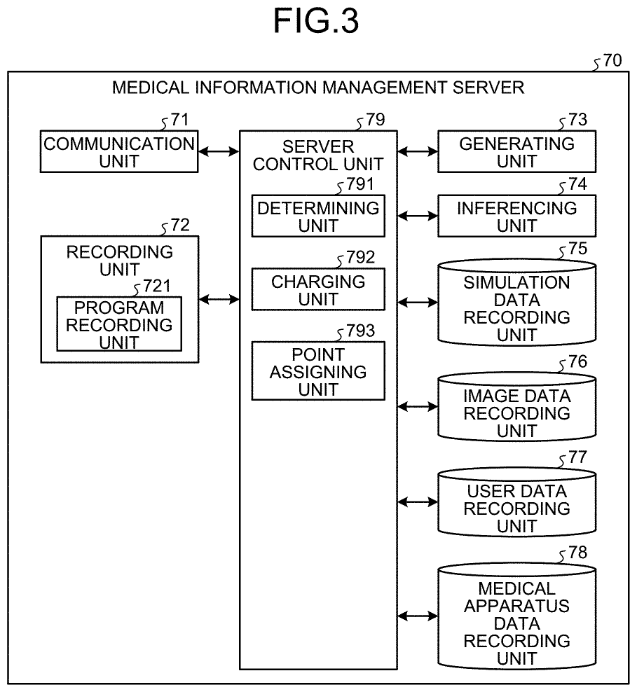

Given below is the explanation of a configuration of the medical information management server 70. FIG. 3 is a block diagram illustrating a functional configuration of the medical information management server 70. The medical information management server 70 illustrated in FIG. 3 includes a communication unit 71, a recording unit 72, a generating unit 73, an inferencing unit 74, a simulation data recording unit 75, an image data recording unit 76, a user data recording unit 77, a medical apparatus data recording unit 78, and a server control unit 79.

Under the control of the server control unit 79, the communication unit 71 sends the simulation data, which is requested by the control device 60 (the client side) via the network 90, to the control device 60. The communication unit 71 is implemented using a communication module.

The recording unit 72 is used to record a variety of data related to the medical information management server 70. The recording unit 72 includes a program recording unit 721 that is used to record various programs executed by the medical information management server 70. The recording unit 72 is implemented using a RAM and a ROM.

Under the control of the server control unit 79, the generating unit 73 generates simulation data at least either based on the intra-operative video data that is sent by a plurality of hospital management servers 80 via the network 90 and that is captured during the surgeries of the subjects or based on the two-dimensional image data and the three-dimensional image data captured in advance from the subjects using CT devices or MRI devices; and records the simulation data in the simulation data recording unit 75. Moreover, based on the inference result obtained by the inferencing unit 74 (described later), the generating unit 73 performs position adjustment of the internal organs captured in at least either the intra-operative video data or the two-dimensional image data and the three-dimensional image data, and generates the simulation data. Furthermore, based on the inference result obtained by the inferencing unit 74 (described later), the generating unit 73 generates simulation data in which the orientation and the extent of collapse of the internal organs, which are captured in at least either the intra-operative video data or the two-dimensional image data and the three-dimensional image data, is reflected. The generating unit 73 is implemented using a processor that includes hardware such as a memory and a GPU.

The inferencing unit 74 estimates the internal organ of the subject as captured in the intra-operative video data and the position of the internal organ at least either based on the intra-operative video data sent by each of a plurality of hospital management servers 80 via the network 90 or based on the two-dimensional image data and the three-dimensional image data; and outputs the estimation result to the generating unit 73. Regarding the inferencing unit 74, the following information is used as the learning parameters (teacher data): the intra-operative video data; the internal organ and its position captured in the intra-operative data; distance information indicating the distance from the leading end of the medical imaging device (videoscope) to the internal organ; the feature quantity of the intra-operative video data; and a plurality of sets of simulation data (for example, polygonal data based on at least either CT image data and MRI image data). Then, the learning parameters are machine-learnt using deep learning, and the inferencing unit 74 is implemented using an already-learnt model that outputs, as output parameters, the estimation result of estimating the target internal organ for observation and its position captured in the intra-operative video data. Herein, in machine learning, it is possible to use a deep neural network (DNN), a convolutional neural network (CNN), or a recurrent neural network (RNN). Moreover, if the intra-operative video data sent by each of a plurality of hospital management servers 80 via the network 90 is multispectral video data obtained as a result of irradiation of multispectral light by a light source device, then the inferencing unit 74 makes use of the difference in the reflected-light frequencies (in each spectrum) as included in the multispectral image data and estimates the body tissue type of the target part for observation (for example, the internal organ such as the gallbladder or the pancreas, the blood vessels, the nerves, the lymph, the urinary duct, and the fat) and its position (area); and outputs the estimation result to the generating unit 73. Moreover, based on the positions and the orientations of the medical imaging devices associated to the intra-operative video data sent by a plurality of hospital management servers 80 via the network 90, based on the positions of insertion in the subjects, and based on the amounts of operation (distances) of insertion in the subjects; the inferencing unit 74 estimates the orientation (direction) and the extent of collapse of the internal organ, the blood vessels, and the nerves captured in the intra-operative video data with reference to the optical axis of the optical system of the medical imaging devices (i.e. with reference to the direction of insertion of the trocars); and outputs the estimation result to the generating unit 73. The inferencing unit 74 is implemented using a processor that includes hardware such as a memory and a GPU.

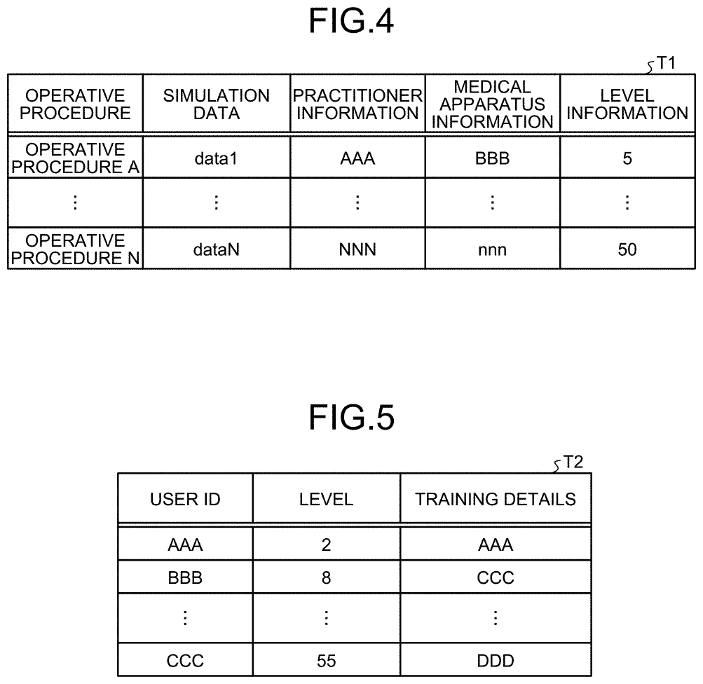

The simulation data recording unit 75 is used to store a plurality of operative procedures in a corresponding manner to a plurality of sets of simulation data. More particularly, as illustrated in Table T1 in FIG. 4, the simulation data recording unit 75 is used to record the following information in a corresponding manner: the operative procedures; the simulation data; practitioner information enabling identification of the practitioners who performed surgery corresponding to the simulation data; medical apparatus information related to the medical apparatuses used in the simulation data; and level information indicating the levels of the simulation data. The simulation data recording unit 75 is implemented using an HDD or an SSD.

The image data recording unit 76 is used to record, in a corresponding manner, two-dimensional image data, which is captured using CT devices, or three-dimensional image data, which is captured using the MRI device, sent from a plurality of hospital management servers 80 via the network 90; hospital identification information indicating the hospital names; and subject information (patient information) related to the subjects. Herein, the subject information indicates the name of the subject, the hospital name, the diagnosis history, the biological information, the primary doctor, and the operating surgeon. The image data recording unit 76 is implemented using an HDD or an SSD.

The user data recording unit 77 is used to record identification information related to each of a plurality of practitioners U1 who took the training using the surgery training device 2 in the medical information management server 70 via the network 90. More particularly, as illustrated in a Table T2 in FIG. 5, the user data recording unit 77 is used to record the following information in a corresponding manner as the identification information enabling identification of the operators U1: user IDs, levels (levels of difficulty) of the surgery training of the operators U1, and training details. Moreover, in the user data recording unit 77, in a corresponding manner to the user IDs; it is also possible to record the training counts, the hospitals or the academic societies to which the user IDs belong, the addresses, and the bank account details. The user data recording unit 77 is implemented using an HDD or an SSD.

The medical apparatus data recording unit 78 is used to record the medical apparatus information related to the medical apparatuses. Herein, the medical apparatus information indicates the following information of a medical apparatus: model name, part number, animation data, and CAD data (polygonal data).

The server control unit 79 controls the constituent elements of the medical information management server 70. The server control unit 79 is implemented using a processor that includes hardware such as a memory and a CPU. When one of a plurality of operative procedures is selected in the control device 60 (the client side), the server control unit 79 sends simulation data corresponding to the selected operative procedure to the control device 60 (the client side). The server control unit 79 includes a determining unit 791, a charging unit 792, and a point assigning unit 793. Meanwhile, according to the embodiment, the server control unit 79 functions as a first control unit.

The determining unit 791 determines about the identification information of the operator U1 who accessed the medical information management server 70, determines about the identification information recorded in the user data recording unit 77, and outputs the determination result to the charging unit 792 and the point assigning unit 793.

Based on the determination result obtained by the determining unit 791, the charging unit 792 charges the registered address or the affiliated hospital, which is registered by the user having the user ID recorded in the user data recording unit 77, a fee based on the level or the downloading count of the simulation data downloaded in the control device 60.

Based on the determination result obtained by the determining unit 791, the point assigning unit 793 assigns points to the user ID, which is recorded in the user data recording unit 77, according to the level associated to the simulation video or according to the number of times for which the training was taken using the simulation data.

Overview of Training Method

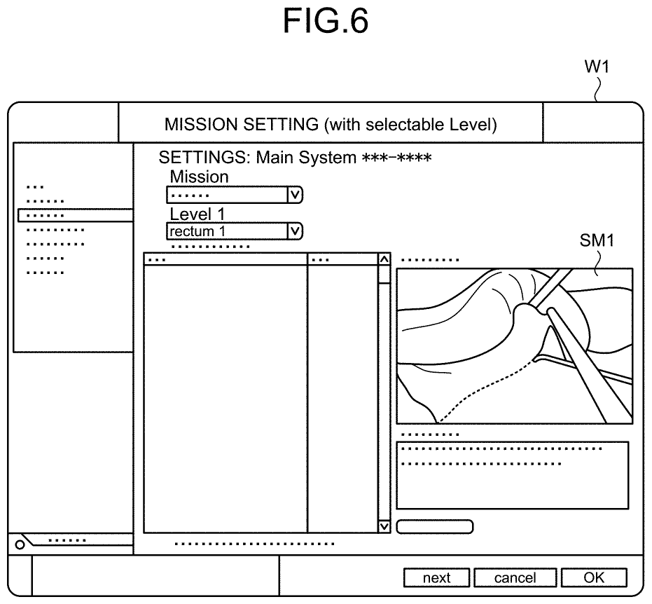

Given below is the explanation of the overview of a surgery training method implemented in the surgery training system 1. FIGS. 6 to 11 are diagrams illustrating examples of setting screens displayed in the display device 50 during the surgery training.

Firstly, the operator U1 accesses the browser of the medical information management server 70 using the input unit 63 of the control device 60, and inputs the identification information (a user ID and a password) enabling his or her identification. In that case, the medical information management server 70 sends training screen data to the control device 60 (the client side) from which the browser was accessed. As a result, as illustrated in FIG. 6, under the control of the control device 60, the display device 50 displays a training screen W1 corresponding to the training screen data. The training screen W1 includes at least the level of the operative procedure, the surgery target (for example, the rectum) in the operative procedure, the mission (for example, removal of a tumor and suturation), and a thumbnail image SM1 corresponding to simulation data. As illustrated in FIG. 6, the operator U1 operates a pointer using the input unit 63 and sets the desired level and the desired surgery target in predetermined cells. In that case, the control device 60 sends the setting details, which are set by the operator U1, to the medical information management server 70.

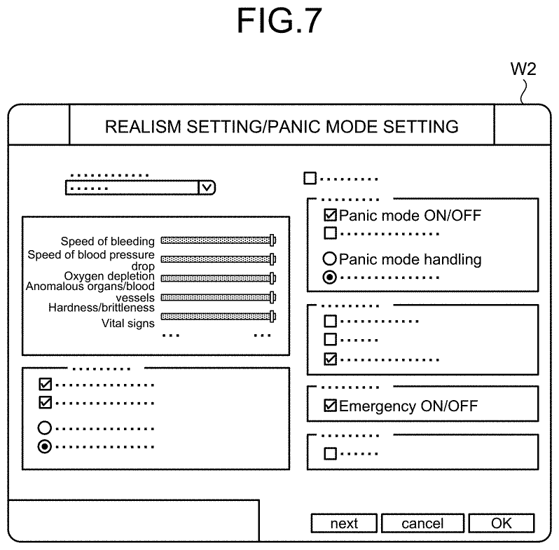

Moreover, as illustrated in FIG. 7, when a request signal regarding realism setting of the operative procedure and requesting setting of various modes during the training is received from the control device 60, the medical information management server 70 sends realism setting/panic mode setting screen data to the control device 60. As a result, as illustrated in FIG. 7, under the control of the control device 60, the display device 50 displays the realism setting/panic mode setting screen W2. The realism setting/panic mode setting screen W2 includes at least the speed of bleeding, the speed of blood pressure drop, oxygen depletion, anomalous organs/blood vessels, hardness/brittleness, vital signs, and the presence/absence of panic during the surgery.

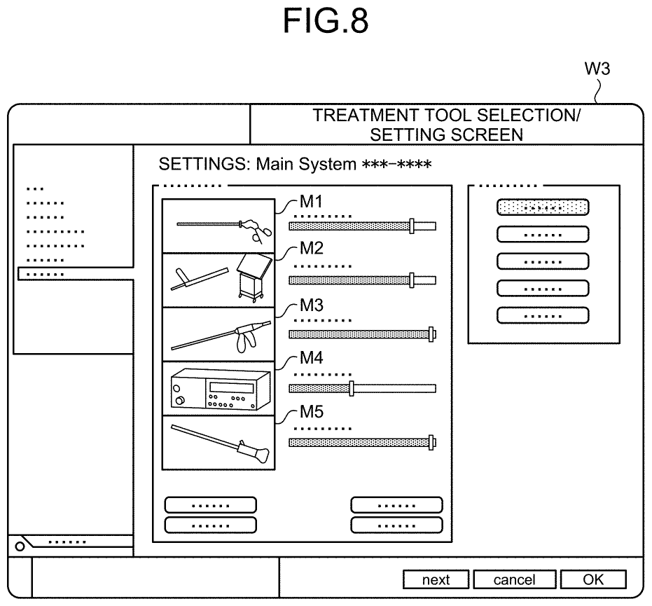

Furthermore, as illustrated in FIG. 8, when a request signal regarding selection and setting of medial apparatuses is received from the control device 60, the medical information management server 70 sends treatment tool selection/setting screen data to the control device 60. As a result, as illustrated in FIG. 8, under the control of the control device 60, the display device 50 displays a treatment tool selection/setting screen W3. The treatment tool selection/setting screen W3 includes a plurality of medical apparatuses M1 to M5 (a plurality of treatment tools) to be used in the operative procedure selected by the operator U1. For example, the treatment tool selection/setting screen W3 includes forceps, an electrocautery scalpel, a medical imaging device (a rigid endoscope), and the like. Thus, the operator U1 selects the medical apparatuses to be used in the operative procedure during the training. Moreover, the operator U1 may pay a fee to the medical information management server 70 and increase the selectable medical apparatuses (selectable items). In that case, based on the identification information of the operator U1, the medical information management server 70 charges a fee according to the number of selected medical apparatuses.

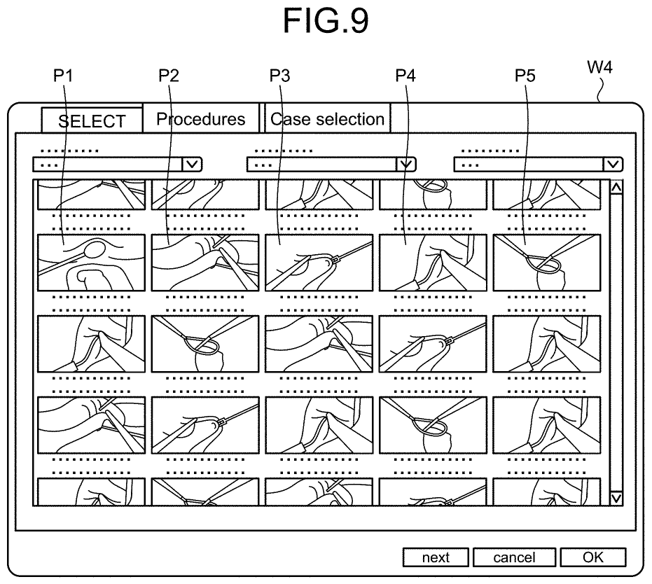

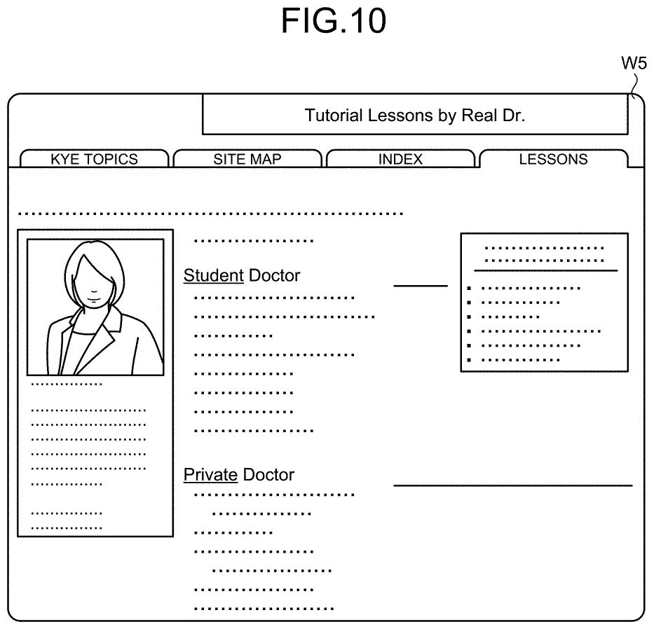

Moreover, as illustrated in FIG. 9, when a request signal regarding an operative procedure/case database is received from the control device 60, the medical information management server 70 sends, to the control device 60, case selection screen data in which thumbnail images of a plurality of sets of simulation data corresponding a plurality of operative procedures or a plurality of cases is listed. As a result, as illustrated in FIG. 9, under the control of the control device 60, the display device 50 displays a case selection screen W4. The case selection screen W4 includes thumbnail images P1 to P5 corresponding to a plurality of sets of simulation data. The operator U1 selects the desired thumbnail image. In that case, the medical information management server 70 sends, to the control device 60, practitioner data related to the practitioner (operating surgeon) who performed the surgery corresponding to the simulation data of the thumbnail image related to the selection signal input from the control device 60. As a result, as illustrated in FIG. 10, under the control of the control device 60, the display device 50 displays a practitioner screen W5 related to the practitioner such as a preceptor. The practitioner screen W5 includes an image of the practitioner (operating surgeon), the case for which the practitioner performed the surgery, the operative procedure, the level according to the surgery training system 1, and a message.

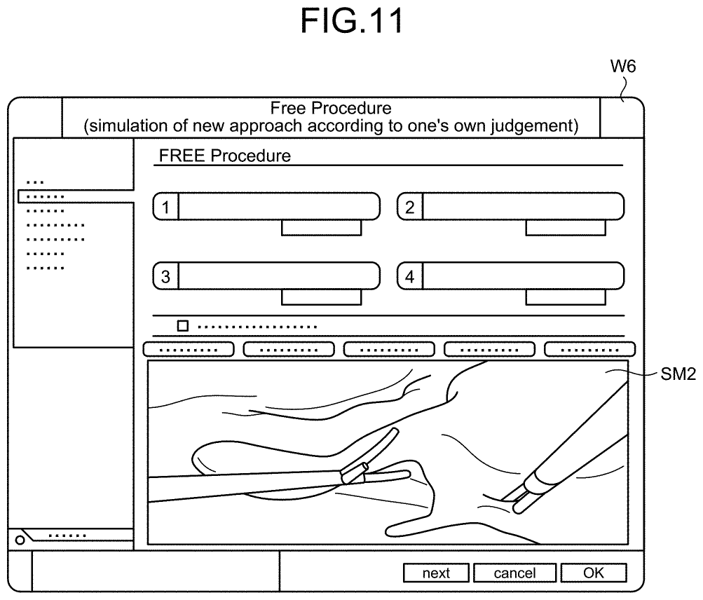

Furthermore, as illustrated in FIG. 11, when a request signal regarding simulation data of a new approach is received from the control device 60, the medical information management server 70 sends simulation screen data of the new approach to the control device 60. As a result, as illustrated in FIG. 11, under the control of the control device 60, the display device 50 displays a simulation data screen W6 of the new approach. The simulation data screen W6 includes a thumbnail image SM2 corresponding to the simulation video.

In this way, using the input unit 63 of the control device 60, the operator U1 may take training by downloading, from the medical information management server 70 in the control device 60, the simulation data of the desired surgical case appropriate to his or her level.

Operations Performed in Medical Information Management Server

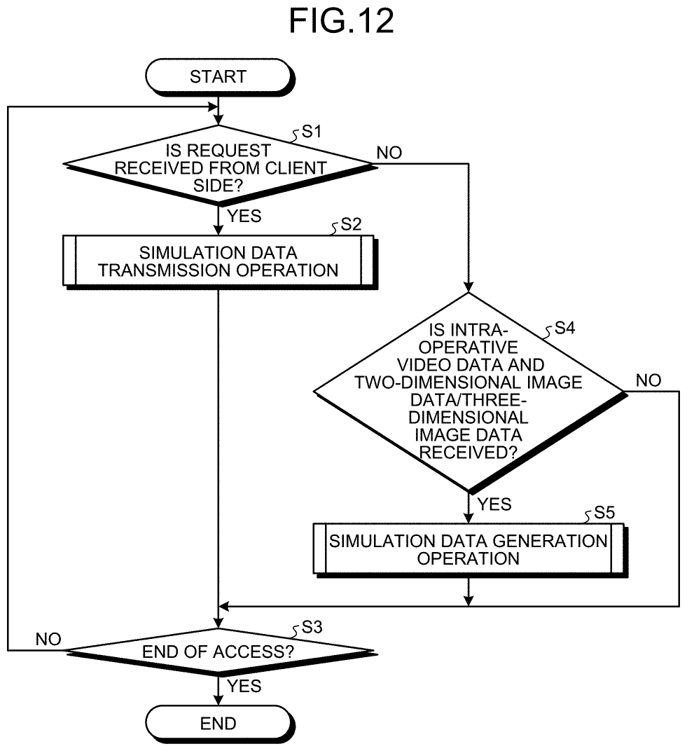

Given below is the explanation of the operations performed in the medical information management server 70. FIG. 12 is a flowchart for explaining the overview of the operations performed in the medical information management server 70.

Firstly, the server control unit 79 determines whether or not a request is received from the client side (Step S1). More particularly, the server control unit 79 determines whether or not a request signal regarding a variety of simulation data is input from the control device 60 (the client side) via the network 90. If the server control unit 79 determines that a request is received from the client side (Yes at Step S1), then the system control proceeds to Step S2 (described later). On the other hand, if the server control unit 79 determines that no request is received from the client side (No at Step S1), then the system control proceeds to Step S4 (described later).

At Step S2, the medical information management server 70 performs a simulation data transmission operation for sending simulation data to the client side. Regarding the simulation data transmission operation, the detailed explanation is given later.

Then, the server control unit 79 determines whether or not the access from the client side has ended (Step S3). If the server control unit 79 determines that the access from the client side has ended (Yes at Step S3), then the medical information management server 70 ends the operations. On the other hand, if the server control unit 79 determines that the access from the client side has not ended (No at Step S3), then the system control returns to Step S1 explained earlier.

At Step S4, the server control unit 79 determines whether or not intra-operative video data and two-dimensional image data/three-dimensional image data are received from any one of a plurality of hospital management servers 80. If the server control unit 79 determines that intra-operative video data and two-dimensional image data/three-dimensional image data are received from any one of a plurality of hospital management servers 80 (Yes at Step S4), then the system control proceeds to Step S5 (described later). On the other hand, if the server control unit 79 determines that intra-operative video data and two-dimensional image data/three-dimensional image data are not received from any one of a plurality of hospital management servers 80 (No at Step S4), then the system control returns to Step S3.

At Step S5, based on either the intra-operative video data or the two-dimensional image data and the three-dimensional video data, the medical information management server 70 performs a simulation data generation operation for generating simulation data. Regarding the simulation data generation operation, the detailed explanation is given later. After the operation at Step S5 is performed, the system control returns to Step S3.

Overview of Simulation Data Transmission Operation

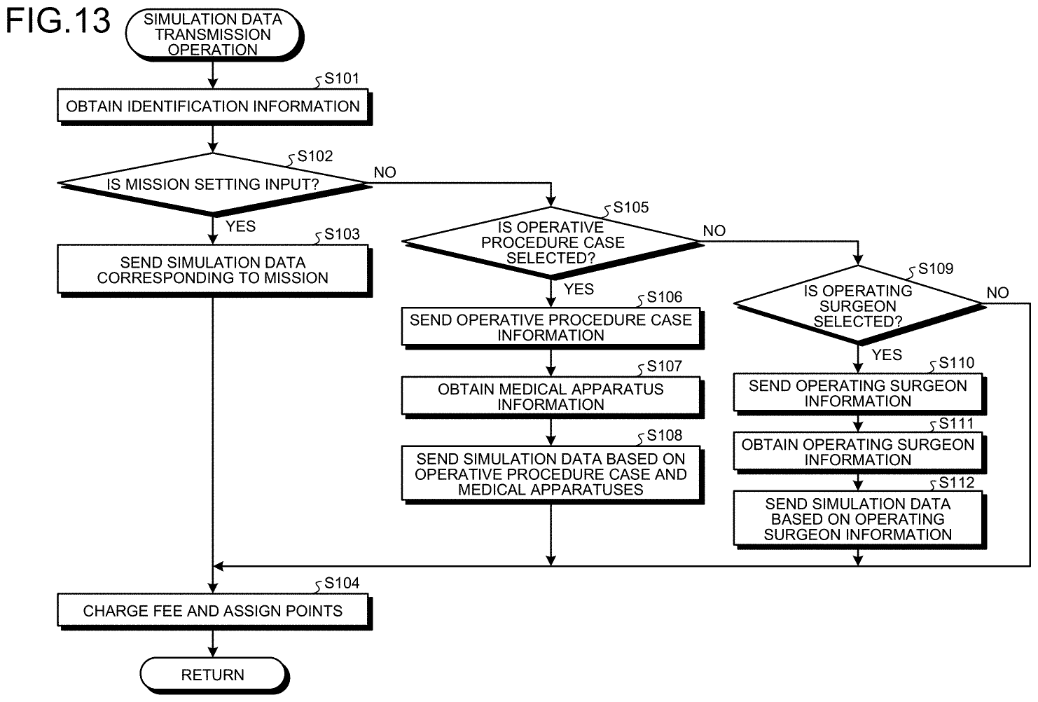

Given below is the explanation of the overview of the simulation data transmission operation explained earlier at Step S2 illustrated in FIG. 12. FIG. 13 is a flowchart for explaining the overview of the simulation data transmission operation.

As illustrated in FIG. 13, firstly, the server control unit 79 obtains identification information, which enables identification of the operator U1 and which is sent from the control device 60 (the client side) accessed via the network 90 and the communication unit 71 (Step S101). In that case, the determining unit 791 determines about the identification information recorded in the user data recording unit 77 and determines about the identification information of the operator U1 sent from the control device 60.

Then, the determining unit 791 determines whether or not a setting signal for setting a mission during the surgery training is input from the control device 60 (Step S102). If it is determined that a setting signal for setting a mission during the surgery training is input from the control device 60 (Yes at Step S102), then the system control proceeds to Step S103 (described later). On the other hand, if a setting signal for setting a mission during the surgery training is not input from the control device 60 (No at Step S102), then the system control proceeds to Step S105 (described later).

At Step S103, the server control unit 79 makes the communication unit 71 send simulation data that corresponds to the mission set in the surgery training by the control device 60 (Step S103). More particularly, the server control unit 79 obtains, from among a plurality of sets of simulation data stored in the simulation data recording unit 75, the simulation data corresponding to the mission set in the surgery training by the control device 60, and makes the communication unit 71 send the obtained simulation data. As a result, while looking at the simulation video displayed in the display device 50, the operator U1 becomes able to take training according to the set mission.

Subsequently, based on the identification information and based on the level of the simulation data downloaded in the control device 60, the server control unit 79 sets a fee for the operator U1 and assigns points to the identification information (Step S104). More particularly, the charging unit 792 charges the registered address or the affiliated hospital, which is registered by the user who matches with the user ID that is determined by the determining unit 791 and that is recorded in the user data recording unit 77, a fee based on the level or the downloading count of the simulation data downloaded in the control device 60. Moreover, the point assigning unit 793 assigns points to the user ID, which is determined by the determining unit 791 and which is recorded in the user data recording unit 77, according to the level associated to the simulation video or according to the number of times for which the training was taken using the simulation data. After the operation at Step S104 is performed, the system control returns to the main routine illustrated in FIG. 12. Meanwhile, the charging method implemented by the charging unit 792 is only exemplary; and, instead of charging a fee according to the level of the simulation data, it is possible to charge a fee for the download of the medical apparatus information of new medical apparatuses, or for the download of the simulation data of a new operative procedure, or for the download of simulation data corresponding to a predetermined operating surgeon.

At Step S105, the determining unit 791 determines whether or not a request signal regarding selection of the desired operative procedure case from among a plurality of operative procedure cases is input from the control device 60. If the determining unit 791 determines that a request signal regarding selection of the desired operative procedure case from among a plurality of operative procedure cases is input from the control device 60 (Yes at Step S105), then the system control proceeds to Step S106 (described later). On the other hand, if the determining unit 791 determines whether or not a request signal regarding selection of the desired operative procedure case from among a plurality of operative procedure cases is not input from the control device 60 (No at Step S105), then the system control proceeds to Step S109 (described later).

At Step S106, the server control unit 79 makes the communication unit 71 send operative procedure case information, in which a plurality of operative procedure cases is compiled corresponding to a plurality of sets of simulation data recorded in the simulation data recording unit 75.

Then, the server control unit 79 obtains, from the control device 60 via the network 90 and the communication unit 71, the medical apparatus information used by the operator U1 during the surgery training (Step S107).

Subsequently, the server control unit 79 makes the communication unit 71 send simulation data, from among a plurality of sets of simulation data recorded in the simulation data recording unit 75, based on the operative procedure case information and the medical apparatus information selected by the operator U1 (Step S108). As a result, while looking at the simulation video displayed in the display device 50, the operator U1 becomes able to take surgery training of the desired operative procedure case using the medical apparatuses 30 and 40. After the operation at Step S108 is performed, the system control returns to Step S104.

At Step S109, the determining unit 791 determines whether or not a request signal regarding selection of the desired operating surgeon from among a plurality of operating surgeons is input from the control device 60. If the determining unit 791 determines that a request signal regarding selection of the desired operating surgeon from among a plurality of operating surgeons is input from the control device 60 (Yes at Step S109), then the system control proceeds to Step S110 (described later). On the other hand, if the determining unit 791 determines that a request signal regarding selection of the desired operating surgeon from among a plurality of operating surgeons is not input from the control device 60 (No at Step S109), then the system control returns to Step S104.

At Step S110, the server control unit 79 makes the communication unit 71 send operating surgeon information, in which a plurality of operating surgeons is compiled corresponding to a plurality of sets of simulation data recorded in the simulation data recording unit 75.

Subsequently, the server control unit 79 obtains the operating surgeon information, which is selected by the operator U1, from the control device 60 via the network 90 and the communication unit 71 (Step S111); and makes the communication unit 71 send the simulation data that, from among a plurality of sets of simulation data recorded in the simulation data recording unit 75, is based on the operating surgeon information selected by the operator U1 (Step S112). As a result, while looking at the simulation video display in the display device 50, the operator U1 becomes able to trace the operative procedure carried out by the operating surgeon and learn from it. After the operation at Step S112 is performed, the system control proceeds to Step S104.

Overview of Simulation Data Generation Operation

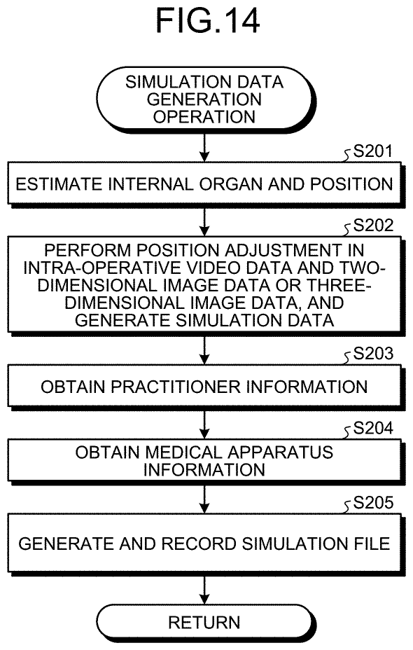

Given below is the explanation of the overview of the simulation data generation operation explained at Step S5 illustrated in FIG. 12. FIG. 14 is a flowchart for explaining the overview of the simulation data generation operation.

As illustrated in FIG. 14, firstly, at least either based on the intra-operative video data sent by each of a plurality of hospital management servers 80 via the network 90 or based on the two-dimensional image data and the three-dimensional image data at least containing data related to the internal organ, the inferencing unit 74 estimates the internal organ of the subject and the position of that internal organ as captured in the intra-operative video data (Step S201). More particularly, if the intra-operative video data sent by each of a plurality of hospital management servers 80 via the network 90 is multispectral video data obtained as a result of irradiation of multispectral light by a light source device, then the inferencing unit 74 makes use of the difference in the reflected-light frequencies (in each spectrum) as included in the multispectral image data and estimates the body tissue type of the target part for observation (for example, the internal organ such as the gallbladder or the pancreas, the blood vessels, the nerves, the lymph, the urinary duct, and the fat) and its position (areas); and outputs the estimation result to the generating unit 73. Moreover, based on the positions and the orientations of the medical imaging devices associated to the intra-operative video data sent by a plurality of hospital management servers 80 via the network 90, based on the positions of insertion in the subjects, and based on the amounts of operation (distances) of insertion in the subjects; the inferencing unit 74 may estimate the orientation (direction) and the extent of collapse of the internal organs, the blood vessels, and the nerves captured in the intra-operative video data with reference to the optical axis of the optical system of the medical imaging devices; and may output the estimation result to the generating unit 73.

Then, based on the inference result obtained by the inferencing unit 74, the generating unit 73 performs position adjustment of the internal organ captured in at least either the intra-operative video data or the two-dimensional image data and the three-dimensional image data, and generates simulation data (Step S202). More particularly, based on the inference result obtained by the inferencing unit 74, after setting the orientation of the internal organ, setting the position of the internal organ, and identifying the internal organ captured in at least either the intra-operative video data or the two-dimensional image data and the three-dimensional image data, the generating unit 73 performs the position adjustment of the internal organ and at the same time generates simulation data in which the orientation and the extent of collapse of the internal organ assumed at the current viewpoint is reflected.

Subsequently, the server control unit 79 obtains practitioner information from metadata (header information) included in an intra-operative video file in which the intra-operative video data is stored (Step S203), and obtains medical apparatus information from the metadata (header information) included in the intra-operative video file in which the intra-operative video data is stored (Step S204).

Then, the server control unit 79 generates a simulation file in which the simulation data generated by the generating unit 73, the practitioner information, and the medical apparatus information are held in a corresponding manner; and records the simulation file in the simulation data recording unit 75 (Step S205). After the operation at Step S205 is performed, the system control returns to the main routine illustrated in FIG. 12.

Operations Performed in Surgery Training Device

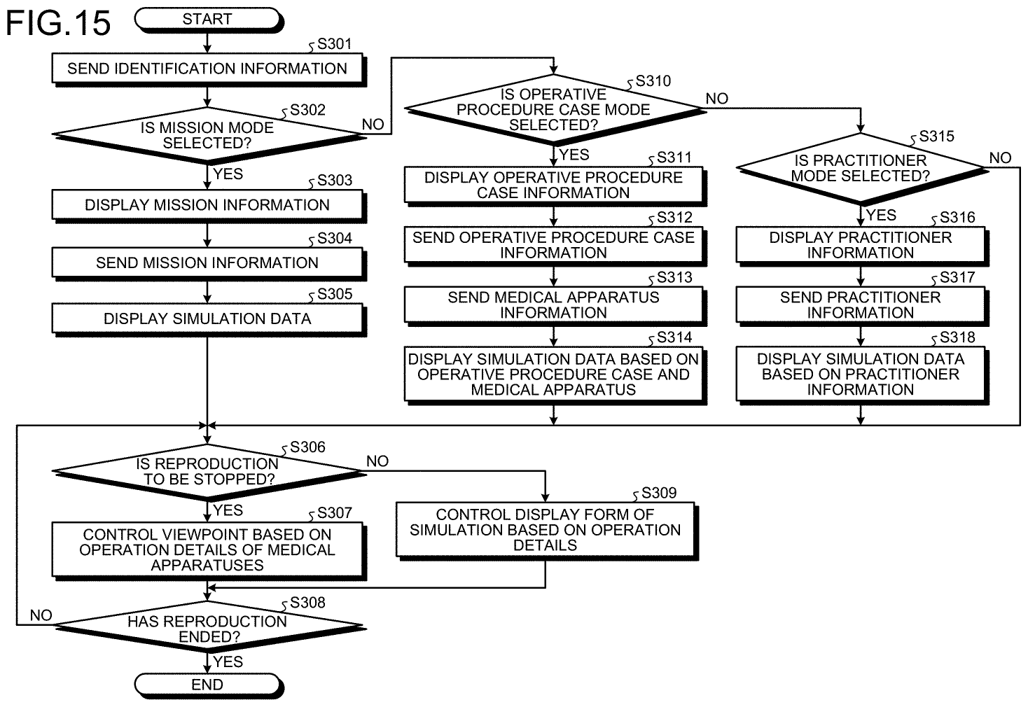

Given below is the explanation of the operations performed in the surgery training device 2. FIG. 15 is a flowchart for explaining the overview of the operations performed in the surgery training device 2.

Firstly, the control unit 66 makes the second communication unit 65 send the identification information of the operator U1 that is input via the input unit 63 (Step S301).

Then, if the operator U1 has selected a mission mode via the input unit 63 (Yes at Step S302), then the control unit 66 displays mission information, which may be set in the surgery training, in the display device 50 (Step S303).

Subsequently, the control unit 66 makes the second communication unit 65 send the mission information that is set by the operator U1 using the input unit 63 (Step S304).

Then, the control unit 66 displays, in the display device 50, a simulation video based on the simulation data corresponding to the mission that is set by the operator U1 and that is sent from the medical information management server 70 (Step S305).

Then, the control unit 66 determines whether or not a termination signal for stopping the reproduction of the simulation video is input from the input unit 63 (Step S306). If the control unit 66 determines that a termination signal for stopping the reproduction of the simulation video is input from the input unit 63 (Step S306), then the system control proceeds to Step S307 (described below). On the other hand, if the control unit 66 determines that a termination signal for stopping the reproduction of the simulation video is not input from the input unit 63 (No at Step S306), then the system control proceeds to Step S309 (described later).

At Step S307, the control unit 66 controls the viewpoint of the simulation video based on the operation details of the medical apparatuses 30 and 40. More particularly, based on the operation details of the medical apparatuses 30 and 40, the control unit 66 controls the image processing unit 62 to generate, as the viewpoint of the simulation video, an image of the display layer on the lower side of the image of the current display layer or an image of the display layer that is observable when the viewpoint is rotated by 90° to the right side from the current viewpoint; and thus controls the viewpoint of the simulation video displayed in the display device 50. Of course, based on the operation details of the medical apparatuses 30 and 40, the control unit 66 may control the image processing unit 62 and perform, for example, magnification and reduction, apart from changing the viewpoint of the simulation video.

Subsequently, when the reproduction of the simulation video ends (Yes at Step S308), the surgery training device 2 ends the operations. On the other hand, if the reproduction of the simulation video has not ended (No at Step S308), then the system control returns to Step S306.

At Step S309, based on the operation details of the medical apparatuses 30 and 40, the control unit 66 controls the display form of the simulation video that is based on the simulation data. More particularly, based on the operation details of the medical apparatuses 30 and 40, the control unit 66 moves the medical apparatuses 30 and 40, which appear in the simulation video, according to the operation details of the operator U1, and reflects the procedure performed using the medical apparatuses 30 and 40 in the simulation video. After the operation at Step S309 is performed, the system control returns to Step S308.

Given below is the explanation about the case in which, at Step S302, the operator U1 has not selected a mission mode via the input unit 63 (No at Step S302). In that case, the system control proceeds to Step S310.

Subsequently, if the operator U1 has selected an operative procedure case mode (Yes at Step S310), then the system control proceeds to Step S311. On the other hand, if the operator U1 has not selected the operative procedure case mode (No at Step S310), then the system control proceeds to Step S315 (described later).

At Step S311, the control unit 66 displays, in the display device 50, operative procedure case information that is selectable during surgery training.

Then, the control unit 66 makes the second communication unit 65 send the operative procedure case information that is selected by the operator U1 using the input unit 63 (Step S312).

Subsequently, the control unit 66 makes the second communication unit 65 send the medical apparatus information related to the medical apparatuses 30 and 40 (Step S313).

Then, the control unit 66 displays, in the display device 50, a simulation video based on the simulation data corresponding to the operative procedure case that is selected by the operator U1 and that is sent from the medical information management server 70 (Step S314). After the operation at Step S314 is performed, the system control returns to Step S306.

At Step S315, if the operator U1 selects a practitioner mode via the input unit 63 (Yes at Step S315), then the system control proceeds to Step S316 (described below).

At Step S316, the control unit 66 displays, in the display device 50, practitioner information that enables selection of a plurality of practitioners in the surgery mode.

Then, the control unit 66 makes the second communication unit 65 send the practitioner information selected by the operator U1 via the input unit 63 (Step S317).

Subsequently, the control unit 66 displays, in the display device 50, a simulation video based on the simulation data corresponding to the practitioner information selected by the operator U1 and sent from the medical information management server 70 (Step S318). After the operation at Step S318 is performed, the system control returns to Step S306.

According to the embodiment explained above, when one of a plurality of operative procedures is selected in the control device 60 (the client side), the server control unit 79 sends the simulation data corresponding to the selected operative procedure. That enables the operator U1 to gain experience equivalent to actual surgeries, so that there is enhancement in the learning curve of the operator U1.

Moreover, according to the embodiment, the training of the operative procedures may be taken in free time regardless of the place.

Furthermore, according to the embodiment, the generating unit 73 generates simulation data based on: the intra-operative video data that is captured by the medical imaging device during the surgery of the subject and that is sent from each hospital management server 80; and at least either two-dimensional image data or three-dimensional image data containing data related to at least an internal organ of the subject captured in advance using a CT device or an MRI device. Hence, the simulation data of a plurality of operative procedures and a plurality of cases may be collected with ease.

Moreover, according to the embodiment, based on the inference result obtained by the inferencing unit 74, the generating unit 73 generates simulation data by performing position adjustment of the internal organ included in at least either the two-dimensional image data or the three-dimensional image data. As a result, it becomes possible to generate realistic simulation data.

Furthermore, according to the embodiment, since the simulation data recording unit 75 is used to record a plurality of sets of simulation data in a corresponding manner to a plurality of sets of practitioner information related to a plurality of practitioners who were the operating surgeons corresponding to the sets of simulation data. Hence, the operator U1 becomes able to take training by selecting the simulation data corresponding to the desired practitioner.

Moreover, according to the embodiment, the simulation data recording unit 75 is used to record a plurality of sets of simulation data in a corresponding manner also to a plurality of sets of medical apparatus information related to a plurality of medical apparatuses used in each of a plurality of sets of simulation data. Hence, the operator U1 becomes able to take training by selecting the simulation data in which the desired medical apparatuses are used.

Furthermore, according to the embodiment, when one or more sets of a plurality of sets of medical apparatus information are selected in the control device 60 (the client side), the server control unit 79 sends the selected medical apparatus information to the control device 60. Hence, the operator U1 becomes able to take training with the simulation data in which the desired medical apparatuses are used.

Moreover, according to the embodiment, when the simulation data is sent to the control device 60 (the client side), the charging unit 792 of the server control unit 79 charges the operator U1 a fee based on the identification information of the operator U1 who operates the client side. As a result, it becomes possible to make a profit.

Furthermore, according to the embodiment, based on the level associated to the identification information of the operator U1 as sent from the control device 60; the server control unit 79 sends, to the control device 60, the operative procedure information related to operative procedures selectable in the control device 60. Hence, the operator U1 becomes able to select the simulation data suitable to his or her level.

Moreover, according to the embodiment, based on the identification information of the operator U1, the server control unit 79 virtually assigns points with respect to the number of times for which the training was taken using the simulation data. Hence, it becomes possible to easily understand the level of the procedures performed by the operator U1.

Furthermore, according to the embodiment, the simulation data recording unit 75 is used to store a plurality of sets of simulation data also corresponding to the levels thereof. Hence, the operator U1 becomes able to take training by selecting the simulation data according to his or her own level.

Moreover, according to the embodiment, based on the operation details of the operator U1 as detected by the first detecting unit 31 and the second detecting unit 32, the control unit 66 controls the display form of the medical apparatus images and the simulation video displayed in the display device 50. Hence, it becomes possible to perform operations in an intuitive manner.

Furthermore, according to the embodiment, when a termination signal for stopping the simulation video being displayed in the display device 50 is input from the input unit 63, based on the operation details detected by the first detecting unit 31 and the second detecting unit 32, the control unit 66 changes the viewpoint of the simulation video, thereby enabling the operator U1 to observe the simulation video from the desired viewpoint.

Moreover, according to the embodiment, the control unit 66 displays, in the display device 50, a plurality of sets of operative procedure information related to a plurality of operative procedures sent from the medical information management server 70 and, when one of the sets of operative surgery information is selected via the input unit 63, requests the medical information management server 70 to send the simulation data corresponding to the selected operative procedure information. As a result, the operator U1 becomes able to obtain the simulation data corresponding to the desired case from the medical information management server 70.

Furthermore, according to the embodiment, since the control unit 66 sends the identification information of the operator U1 to the medical information management server 70, the simulation data corresponding to the operator U1 may be obtained.

Moreover, according to the embodiment, the control unit 66 sends, to the medical information management server 70, the medical apparatus information related to the medical apparatuses 30 and 40 to be used by the operator U1. Hence, the operator U1 becomes able to obtain the simulation data suitable to the medical apparatuses 30 and 40 to be used in the training.

Meanwhile, according to the embodiment, although only a single operator U1 is taking training, that is not the only possible case. Alternatively, a plurality of operators U1 may take training while looking at the same simulation video displayed in the display device 50, or a plurality of operators U1 may take training using the surgery training devices installed at mutually different locations.

### Other Embodiments

A plurality of constituent elements disclosed in the surgery training system according to the embodiment of the disclosure may be appropriately combined to form variations. For example, some of the constituent elements of the surgery training system according to the embodiment of the disclosure may be deleted. Moreover, the constituent elements of the medical observation system according to the embodiment of the disclosure may be appropriately combined.

the term “unit” mentioned above may be read as “device” or “circuit”. For example, a control unit may be read as a control device or a control circuit.

Meanwhile, a computer program executed in the surgery training system according to the embodiment of the disclosure is recorded as installable file data or executable file data in a computer-readable recording medium such as a compact disk read only memory (CD-ROM), a flexible disk (FD), a compact disk recordable (CD-R), a digital versatile disk (DVD), a universal serial bus (USB) medium, or a flash memory.

Alternatively, the computer program executed in the surgery training system according to the embodiment of the disclosure may be stored in a downloadable manner in a computer connected to a network such as the Internet.

Meanwhile, in the explanation of the flowcharts given in the present written description, the context is explicitly illustrated using expressions such as “firstly”, “then”, and “subsequently”. However, the sequence of operations to implement the present disclosure is not uniquely fixed by those expressions. That is, the sequence of operations performed in the flowchart given in the present written description may be varied without causing contradiction.

Although the disclosure is described above in detail in the form of an embodiment with reference to the accompanying drawings; the technical scope of the disclosure is not limited to the embodiment described above. That is, the disclosure is to be construed as embodying all modifications such as other embodiments, additions, alternative constructions, and deletions that may occur to one skilled in the art that fairly fall within the basic teaching herein set forth.

Meanwhile, a configuration as explained below also falls within the technical scope of the disclosure.

(1)

A medical information management server including:

a recording unit configured to record at least a plurality of sets of operative procedures in a corresponding manner to a plurality of sets of simulation data containing data related at least to body tissues; and

a control unit configured to, in a case where one of the plurality of sets of operative procedures is selected at a client side, send simulation data corresponding to the selected operative procedure to the client side.

(2)

The medical information management server according to (1), further including a generating unit configured to generate the simulation data based on at least either

intra-operative video data captured during surgery of subject, or

two-dimensional image data and three-dimensional image data captured in advance from the subject using a CT device or an MRI device.

(3)

The medical information management server according to (2), further including an inferencing unit configured to, based on at least either the intra-operative video data or the two-dimensional image data and the three-dimensional image data, estimate internal organ and position thereof in the subject as captured in the intra-operative video data, wherein

based on the inference result obtained by the inferencing unit, the generating unit is configured to generate the simulation data by performing position adjustment of internal organ included in at least either the intra-operative video data or the two-dimensional image data and the three-dimensional image data.

(4)

The medical information management server according to (3), wherein

when the subject is irradiated with multispectral light emitted by a multispectral light source, optical feedback from the subject is received as a result of which the intra-operative video data contains spectral image data that at least contains reflected frequency of each of body tissues, and

using reflected frequency of each of the body tissues as included in the spectral image data, the inferencing unit is configured to estimate at least types and positions of the body tissues included in the spectral image data.

(5)

The medical information management server according to any one of (1) to (4), wherein

the recording unit is configured to record the plurality of sets of simulation data in a corresponding manner also to a plurality of sets of practitioner information related to a plurality of practitioners who performed surgery corresponding to each of the plurality of sets of simulation data, and

in a case where one of the plurality of sets of practitioner information is selected at the client side, the control unit is configured to send the plurality of sets of simulation data corresponding to the selected practitioner information.

(6)

The medical information management server according to any one of (1) to (5), wherein

the recording unit is configured to further store, in a corresponding manner, a plurality of sets of medical apparatus information related to a plurality of medical apparatuses used in regard to each of the plurality of sets of simulation data, and

in a case where sending the simulation data to the client side, the control unit is configured to also send the medical apparatus information associated to concerned simulation data to the client side.

(7)

The medical information management server according to (6), wherein, in a case where one or more of the plurality of sets of medical apparatus information are selected at the client side, the control unit is configured to send the selected medical apparatus information to the client side.

(8)

The medical information management server according to any one of (1) to (7), wherein, in a case where the simulation data is sent to the client side, the control unit is configured to charge operator of the client side a fee based on identification information of the operator.

(9)

The medical information management server according to (8), wherein, based on level associated to the identification information, the control unit is configured to send, to the client side, operative procedure information related to the operative procedure selected at the client side.

(10)

The medical information management server according to (9), wherein, based on the identification information, the control unit is configured to assign points with respect to number of times for which the operator takes virtual training using the simulation data.

(11)

The medical information management server according to any one of (8) to (10), wherein the recording unit is configured to further record, in a corresponding manner, level of each of the plurality of sets of simulation data.

(12)

A surgery training device including:

a display unit configured to display a simulation video based on simulation data sent from a medical information management server which records a plurality of operative procedures in a corresponding manner to a plurality of sets of simulation data containing data related at least to body tissues;

a medical apparatus;

a detecting unit configured to detect operation details of operation performed by an operator with respect to the medical apparatus; and

a control unit configured to display, in the display unit, medical apparatus images, which are related to the medical apparatuses, on the simulation video, wherein

based on the operation details detected by the detecting unit, the control unit is configured to control display form of the medical apparatus images and the simulation video.

(13)

The surgery training device according to (12), wherein, when a termination signal for stopping the simulation video displayed in the display unit is input, in response to detection of the operation details by the detecting unit, the control unit is configured to change viewpoint of the simulation video based on the operation details.

(14)

The surgery training device according to (13), wherein, in a case where a plurality of sets of operative procedure information related to the plurality of operative procedures is displayed in the display unit and one or more of the plurality of sets of operative procedure information are selected, the control unit is configured to request the medical information management server to send the simulation data corresponding to the selected sets of operative procedure information.

(15)

The surgery training device according to (14), further including an input unit configured to receive input of identification information of the operator, wherein

the control unit is configured to send the identification information to the medical information management server.

(16)

The surgery training device according to any one of (12) to (15), wherein the control unit is configured to send medical apparatus information related to the medical apparatus to the medical information management server.

(17)

A surgery training system including:

a medical information management server; and

a surgery training device configured to communicate with the medical information management server via a network, wherein

the medical information management server includes
a recording unit configured to record at least a plurality of sets of operative procedures in a corresponding manner to a plurality of sets of simulation data containing data related at least to body tissues, and
a first control unit configured to, in a case where one of the plurality of sets of operative procedures is selected at a client side, send simulation data corresponding to the selected operative procedure to the client side,

the surgery training device includes
a display unit configured to display a simulation video based on the simulation data sent from the medical information management server,
a medical apparatus configured to enable treatment with respect to a model of a subject,
a detecting unit configured to detect operation details of operation performed by an operator with respect to the medical apparatus, and
a second control unit configured to display, in the display unit, medical apparatus images, which are related to the medical apparatus, on the simulation video, and

based on the operation details detected by the detecting unit, the second control unit is configured to control display form of the medical apparatus images and the simulation video.

(18)

An image transmission method implemented in a medical information management server that includes

a recording unit configured to record at least a plurality of sets of operative procedures in a corresponding manner to a plurality of sets of simulation data containing data related at least to body tissues,

the image transmission method including:

sending, in a case where one of the plurality of sets of operative procedures is selected at a client side, simulation data corresponding to the selected operative procedure to the client side.

(19)

A display method implemented in a surgery training device that includes

a display unit configured to display a simulation video based on simulation data sent from a medical information management server which records a plurality of operative procedures in a corresponding manner to a plurality of sets of simulation data containing data related at least to body tissues, and

a medical apparatus configured to enable treatment with respect to a model of a subject,

the display method including:

detecting operation details of operation performed by an operator with respect to the medical apparatus;

displaying, in the display unit, medical apparatus images, which are related to the medical apparatus, on the simulation video; and

controlling, based on the operation details, display form of the medical apparatus images and the simulation video.

(20)

A non-transitory computer-readable recording medium on which an executable program is recorded, the program causing a processor of a medical information management server to execute, the medical information management server including a recording unit configured to record at least a plurality of sets of operative procedures in a corresponding manner to a plurality of sets of simulation data containing data related at least to body tissues:

sending, in a case where one of the plurality of sets of operative procedures is selected at client side, simulation data corresponding to the selected operative procedure to the client side.

(21)

A non-transitory computer-readable recording medium on which an executable program is recorded, the program causing a processor of a surgery training device to execute, the surgery training device including a display unit configured to display a simulation video based on simulation data sent from a medical information management server which records a plurality of operative procedures in a corresponding manner to a plurality of sets of simulation data containing data related at least to body tissues, and a medical apparatus configured to enable treatment with respect to a model of a subject:

detecting operation details of operation performed with respect to the medical apparatuses by an operator;

displaying, in the display unit, medical apparatus images, which are related to the medical apparatus, on the simulation video; and

controlling, based on the operation details, display form of the medical apparatus images and the simulation video.

According to the disclosure, it is possible to gain experience equivalent to actual surgeries.

Although the disclosure has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A medical information management server comprising:
a recording unit configured to record at least a plurality of sets of operative procedures in a corresponding manner to a plurality of sets of simulation data containing data related at least to body tissues; and
a control unit configured to, in a case where one of the plurality of sets of operative procedures is selected at a client side, send simulation data corresponding to the selected operative procedure to the client side.

2. The medical information management server according to claim 1, further comprising a generating unit configured to generate the simulation data based on at least either
intra-operative video data captured during surgery of subject, or
two-dimensional image data and three-dimensional image data captured in advance from the subject using a CT device or an MRI device.

3. The medical information management server according to claim 2, further comprising an inferencing unit configured to, based on at least either the intra-operative video data or the two-dimensional image data and the three-dimensional image data, estimate internal organ and position thereof in the subject as captured in the intra-operative video data, wherein
based on the inference result obtained by the inferencing unit, the generating unit is configured to generate the simulation data by performing position adjustment of internal organ included in at least either the intra-operative video data or the two-dimensional image data and the three-dimensional image data.

4. The medical information management server according to claim 3, wherein
when the subject is irradiated with multispectral light emitted by a multispectral light source, optical feedback from the subject is received as a result of which the intra-operative video data contains spectral image data that at least contains reflected frequency of each of body tissues, and
using reflected frequency of each of the body tissues as included in the spectral image data, the inferencing unit is configured to estimate at least types and positions of the body tissues included in the spectral image data.

5. The medical information management server according to claim 1, wherein
the recording unit is configured to record the plurality of sets of simulation data in a corresponding manner also to a plurality of sets of practitioner information related to a plurality of practitioners who performed surgery corresponding to each of the plurality of sets of simulation data, and
in a case where one of the plurality of sets of practitioner information is selected at the client side, the control unit is configured to send the plurality of sets of simulation data corresponding to the selected practitioner information.

6. The medical information management server according to claim 1, wherein
the recording unit is configured to further store, in a corresponding manner, a plurality of sets of medical apparatus information related to a plurality of medical apparatuses used in regard to each of the plurality of sets of simulation data, and
in a case where sending the simulation data to the client side, the control unit is configured to also send the medical apparatus information associated to concerned simulation data to the client side.

7. The medical information management server according to claim 6, wherein, in a case where one or more of the plurality of sets of medical apparatus information are selected at the client side, the control unit is configured to send the selected medical apparatus information to the client side.

8. The medical information management server according to claim 1, wherein, in a case where the simulation data is sent to the client side, the control unit is configured to charge operator of the client side a fee based on identification information of the operator.

9. The medical information management server according to claim 8, wherein, based on level associated to the identification information, the control unit is configured to send, to the client side, operative procedure information related to the operative procedure selected at the client side.

10. The medical information management server according to claim 9, wherein, based on the identification information, the control unit is configured to assign points with respect to number of times for which the operator takes virtual training using the simulation data.

11. The medical information management server according to claim 8, wherein the recording unit is configured to further record, in a corresponding manner, level of each of the plurality of sets of simulation data.

12. A surgery training device comprising:
a display unit configured to display a simulation video based on simulation data sent from a medical information management server which records a plurality of operative procedures in a corresponding manner to a plurality of sets of simulation data containing data related at least to body tissues;
a medical apparatus;
a detecting unit configured to detect operation details of operation performed by an operator with respect to the medical apparatus; and
a control unit configured to display, in the display unit, medical apparatus images, which are related to the medical apparatuses, on the simulation video, wherein
based on the operation details detected by the detecting unit, the control unit is configured to control display form of the medical apparatus images and the simulation video.

13. The surgery training device according to claim 12, wherein, when a termination signal for stopping the simulation video displayed in the display unit is input, in response to detection of the operation details by the detecting unit, the control unit is configured to change viewpoint of the simulation video based on the operation details.

14. The surgery training device according to claim 13, wherein, in a case where a plurality of sets of operative procedure information related to the plurality of operative procedures is displayed in the display unit and one or more of the plurality of sets of operative procedure information are selected, the control unit is configured to request the medical information management server to send the simulation data corresponding to the selected sets of operative procedure information.

15. The surgery training device according to claim 14, further comprising an input unit configured to receive input of identification information of the operator, wherein
the control unit is configured to send the identification information to the medical information management server.

16. The surgery training device according to claim 12, wherein the control unit is configured to send medical apparatus information related to the medical apparatus to the medical information management server.

17. A surgery training system comprising:
a medical information management server; and
a surgery training device configured to communicate with the medical information management server via a network, wherein
the medical information management server includes
a recording unit configured to record at least a plurality of sets of operative procedures in a corresponding manner to a plurality of sets of simulation data containing data related at least to body tissues, and
a first control unit configured to, in a case where one of the plurality of sets of operative procedures is selected at a client side, send simulation data corresponding to the selected operative procedure to the client side,
the surgery training device includes
a display unit configured to display a simulation video based on the simulation data sent from the medical information management server,
a medical apparatus configured to enable treatment with respect to a model of a subject,
a detecting unit configured to detect operation details of operation performed by an operator with respect to the medical apparatus, and
a second control unit configured to display, in the display unit, medical apparatus images, which are related to the medical apparatus, on the simulation video, and
based on the operation details detected by the detecting unit, the second control unit is configured to control display form of the medical apparatus images and the simulation video.

18. An image transmission method implemented in a medical information management server that includes
a recording unit configured to record at least a plurality of sets of operative procedures in a corresponding manner to a plurality of sets of simulation data containing data related at least to body tissues,
the image transmission method comprising:
sending, in a case where one of the plurality of sets of operative procedures is selected at a client side, simulation data corresponding to the selected operative procedure to the client side.

19. A display method implemented in a surgery training device that includes
a display unit configured to display a simulation video based on simulation data sent from a medical information management server which records a plurality of operative procedures in a corresponding manner to a plurality of sets of simulation data containing data related at least to body tissues, and
a medical apparatus configured to enable treatment with respect to a model of a subject,
the display method comprising:
detecting operation details of operation performed by an operator with respect to the medical apparatus;
displaying, in the display unit, medical apparatus images, which are related to the medical apparatus, on the simulation video; and
controlling, based on the operation details, display form of the medical apparatus images and the simulation video.

20. A non-transitory computer-readable recording medium on which an executable program is recorded, the program causing a processor of a medical information management server to execute, the medical information management server including a recording unit configured to record at least a plurality of sets of operative procedures in a corresponding manner to a plurality of sets of simulation data containing data related at least to body tissues:
sending, in a case where one of the plurality of sets of operative procedures is selected at client side, simulation data corresponding to the selected operative procedure to the client side.

21. A non-transitory computer-readable recording medium on which an executable program is recorded, the program causing a processor of a surgery training device to execute, the surgery training device including a display unit configured to display a simulation video based on simulation data sent from a medical information management server which records a plurality of operative procedures in a corresponding manner to a plurality of sets of simulation data containing data related at least to body tissues, and a medical apparatus configured to enable treatment with respect to a model of a subject:
detecting operation details of operation performed with respect to the medical apparatuses by an operator;
displaying, in the display unit, medical apparatus images, which are related to the medical apparatus, on the simulation video; and
controlling, based on the operation details, display form of the medical apparatus images and the simulation video.
